(19) United States
(12) Patent Application Publication
(10) Pub. No.: US 2014/0342937 A1
(43) Pub. Date: Nov. 20, 2014

(54) METHODS FOR DIAGNOSIS AND THERAPEUTIC FOLLOW-UP OF MUSCULAR DYSTROPHIES

(71) Applicants: GENETHON, EVRY (FR); ASSOCIATION INSTITUT DE MYOLOGIE, Paris (FR)

(72) Inventors: Laurence Jeanson-Leh, Morsang Sur Orge (FR); David Israeli, Paris (FR); Fatima Amor, Le Kremlin Bicetre (FR); Thomas Voit, Boullay Les Troux (FR)

(21) Appl. No.: 14/364,338

(22) PCT Filed: Dec. 14, 2012
(86) PCT No.: PCT/EP2012/075665

§ 371 Date: Jun. 11, 2014

(30) Foreign Application Priority Data
Dec. 16, 2011 (FR) 1161862

(51) Int. Cl. C12Q 1/68

(52) U.S. Cl. CPC C12Q 1/6881; C12Q 2600/178; C12Q 2600/16; C12Q 2600/112
USPC 506/9; 435/6.12; 435/6.11; 435/287.2; 506/16

(57) ABSTRACT

The invention relates to the diagnosis, the follow-up and the evaluation of the efficacy of a treatment of a muscular dystrophy by detection of microRNA in a body fluid, in particular in the urine.

### FIELD OF THE INVENTION

The invention relates to the diagnosis, follow-up and evaluation of the efficacy of a treatment of muscular dystrophy by detection of microRNAs in a body fluid, notably in the urine.

### PRIOR ART

Duchenne muscular dystrophy (DMD) and Becker muscular dystrophy (BMD) are caused by mutations or deletions of the gene coding for dystrophin (Muntoni, Torelli et al. 2003). In the first case, where the phenotype is the most severe, dystrophin is completely absent. The DAPC complex (Dystrophin Associated Protein Complex), which allows attachment of the intracellular actin filaments to the extracellular matrix (Le Rumeur, Winder et al.), is also absent. This complex usually protects the membrane of the muscle fibers, which undergo contraction and relaxation. In its absence, the fibers are no longer protected, and in the muscles we observe muscle cells undergoing degeneration and new cells that are evidence of regeneration that tends to counterbalance the phenomenon (Batchelor and Winder 2006). Eventually, regeneration is insufficient and the fibers are replaced by adipose tissue.

Therapeutically, great hopes are currently placed on the exon skipping technique (Cirak, Arechavala-Gomeza et al.; Lu, Yokota et al.). In fact, BMD, which leads to a less serious phenotype, is also due to one or more mutations in the gene coding for dystrophin but the fundamental domains of the protein are conserved:
an N-terminal domain of binding to the actin filaments,
a cysteine-rich C-terminal domain that binds to the DAPC complex.

For DMD patients, it is therefore possible to obtain a Becker phenotype by excluding the exons bearing nonsense mutations within the messenger RNAs (mRNAs) and thus restore the open reading frame. The protein produced, which is shorter, is then partially functional. This strategy is currently under test in several clinical trials.

Regular multidisciplinary medical monitoring makes it possible to evaluate the progression of the pathology and propose management for improving the patient's life. It involves prevention of retractions, supplying technical aids, physical therapy, cardiac monitoring, orthopedics and respiratory assistance. Diagnostic follow-up is performed by, among other things, evaluation of motor functions, by muscle biopsies or by assay of an enzyme secreted in the circulation, creatine kinase (Bushby, Finkel et al.).

Muscle biopsy analysis makes it possible to observe the damaged fibers, smaller fibers being evidence of muscular regeneration, as well as zones of necrosis replaced by adipose tissue. This method has the drawback that it is very invasive for the patient.

Another method consists of determining creatine kinase (CK) in the blood. This enzyme is associated with the energy metabolism present in several types of cells. Increase of its concentration in the blood is evidence of the state of degradation of the muscle fibers. However, this biomarker is not completely reliable as its level also depends on stress, such as physical activity (Nicholson, Morgan et al. 1986). There are other enzymes such as aldolase or lactate dehydrogenase but, as for CK, their abundance is not solely dependent on the pathological state (Lott and Landesman 1984).

Consequently, it appears to be necessary to identify new biomarkers that are more reliable in the context of Duchenne muscular dystrophy, markers that could be determined on the basis of noninvasive samples, such as a urine sample.

MicroRNAs (or miRNAs) are promising biomarkers. They are expressed in all tissues of the body and notably in the skeletal muscle. It is also known that they exist in the “circulating” state in all biological fluids (Weber, Baxter et al.). Recent works in the literature have shown that a signature specific to the Duchenne myopathy exists in the muscle (Cacchiarelli, Martone et al.; Greco, De Simone et al. 2009) and in the serum (Cacchiarelli, Legnini et al.). To date, no study has shown the potential use of miRNAs as markers of muscular dystrophies in the urine. The inventors investigated the profile of the presence of miRNAs in the urine of patients with muscular dystrophy in order to identify a signature specific to disorders of this type.

Moreover, the inventors also investigated new circulating markers of muscular dystrophy.

### SUMMARY OF THE INVENTION

The inventors notably investigated urine samples of DMD patients in order to determine whether miRNAs specific to this disorder could be identified. This work showed that there is a specific signature relating to the abundance of certain miRNAs in the urine of DMD patients relative to the urine of healthy donors.

The finding of such a variation of the expression of one or more miRNAs in a sick individual relative to a healthy individual finds application in the field of diagnosis. The present invention thus relates to the use of at least one miRNA selected from the miRNAs in Table 1, for application of a method for diagnosis of muscular dystrophy. It further relates to the use of one or more of said miRNAs for evaluating the risk of developing or presenting a muscular dystrophy.

TABLE 1

| urinary miRNA | sequence | seq id# |
|---|---|---|
| hsa-let-7a | UGAGGUAGUAGGUUGUAUAGUU | 1 |
| hsa-let-7b | UGAGGUAGUAGGUUGUGUGGUU | 2 |
| hsa-let-7c | UGAGGUAGUAGGUUGUAUGGUU | 3 |
| hsa-let-7d | AGAGGUAGUAGGUUGCAUAGUU | 4 |
| hsa-let-7e | UGAGGUAGGAGGUUGUAUAGUU | 5 |
| hsa-let-7f | UGAGGUAGUAGAUUGUAUAGUU | 6 |
| hsa-let-7g | UGAGGUAGUAGUUUGUACAGUU | 7 |
| hsa-miR-1244 | AAGUAGUUGGUUUGUAUGAGAUGGUU | 8 |
| hsa-miR-139-5p | UCUACAGUGCACGUGUCUCCAGU | 9 |
| hsa-miR-151-5p | UCGAGGAGCUCACAGUCUAGU | 10 |
| hsa-miR-155 | UUAAUGCUAAUCGUGAUAGGGGU | 11 |
| hsa-miR-15a | UAGCAGCACAUAAUGGUUUGUG | 12 |
| hsa-miR-15b | UAGCAGCACAUCAUGGUUUACA | 13 |
| hsa-miR-182 | UUUGGCAAUGGUAGAACUCACACU | 14 |
| hsa-miR-183 | UAUGGCACUGGUAGAAUUCACU | 15 |
| hsa-miR-192* | CUGCCAAUUCCAUAGGUCACAG | 16 |
| hsa-miR-193a-3p | AACUGGCCUACAAAGUCCCAGU | 17 |
| hsa-miR-196b | UAGGUAGUUUCCUGUUGUUGGG | 18 |
| hsa-miR-198 | GGUCCAGAGGGGAGAUAGGUUC | 19 |
| hsa-miR-200b* | CAUCUUACUGGGCAGCAUUGGA | 20 |
| hsa-miR-206 | UGGAAUGUAAGGAAGUGUGUGG | 21 |
| hsa-miR-208 | AUAAGACGAGCAAAAAGCUUGU | 22 |
| hsa-miR-214 | UGCCUGUCUACACUUGCUGUGC | 23 |
| hsa-miR-216b | AAAUCUCUGCAGGCAAAUGUGA | 24 |
| hsa-miR-220 | CCACACCGUAUCUGACACUUU | 25 |
| hsa-miR-224 | CAAGUCACUAGUGGUUCCGUU | 26 |
| hsa-miR-23a | AUCACAUUGCCAGGGAUUUCC | 27 |
| hsa-miR-23b | AUCACAUUGCCAGGGAUUACC | 28 |
| hsa-miR-26b | UUCAAGUAAUUCAGGAUAGGU | 29 |
| hsa-miR-28-5p | AAGGAGCUCACAGUCUAUUGAG | 30 |
| hsa-miR-30d | UGUAAACAUCCCCGACUGGAAG | 31 |
| hsa-miR-30e-3p | UGUAAACAUCCUUGACUGGAAG | 32 |
| hsa-miR-328 | CUGGCCCUCUCUGCCCUUCCGU | 33 |
| hsa-miR-335 | UCAAGAGCAAUAACGAAAAAUGU | 34 |
| hsa-miR-33a* | CAAUGUUUCCACAGUGCAUCAC | 35 |
| hsa-miR-346 | UGUCUGCCCGCAUGCCUGCCUCU | 36 |
| hsa-miR-34c-5p | AGGCAGUGUAGUUAGCUGAUUGC | 37 |
| hsa-miR-373 | ACUCAAAAUGGGGGCGCUUUCC | 38 |
| hsa-miR-376c | GGUGGAUAUUCCUUCUAUGUU | 39 |
| hsa-miR-381 | AGCGAGGUUGCCCUUUGUAUAU | 40 |
| hsa-miR-410 | AAUAUAACACAGAUGGCCUGU | 41 |
| hsa-miR-412 | ACUUCACCUGGUCCACUAGCCGU | 42 |
| hsa-miR-423-5p | UGAGGGGCAGAGAGCGAGACUUU | 43 |
| hsa-miR-433 | AUCAUGAUGGGCUCCUCGGUGU | 44 |
| hsa-miR-484 | UCAGGCUCAGUCCCCUCCCGAU | 45 |
| hsa-miR-487b | AAUCGUACAGGGUCAUCCACUU | 46 |
| hsa-miR-490-3p | CAACCUGGAGGACUCCAUGCUG | 47 |
| hsa-miR-492 | AGGACCUGCGGGACAAGAUUCUU | 48 |
| hsa-miR-493 | UUGUACAUGGUAGGCUUUCAUU | 49 |
| hsa-miR-494 | UGAAACAUACACGGGAAACCUC | 50 |
| hsa-miR-502-3p | AAUGCACCUGGGCAAGGAUUCA | 51 |
| hsa-miR-505* | GGGAGCCAGGAAGUAUUGAUGU | 52 |
| hsa-miR-511 | GUGUCUUUUGCUCUGCAGUCA | 53 |
| hsa-miR-517b | AUCGUGCAUCCCUUUAGAGUGU | 54 |
| hsa-miR-518a-3p | GAAAGCGCUUCCCUUUGCUGGA | 55 |
| hsa-miR-518b | CAAAGCGCUCCCCUUUAGAGGU | 56 |
| hsa-miR-518e | AAAGCGCUUCCCUUCAGAGUG | 57 |
| hsa-miR-518f | GAAAGCGCUUCUCUUUAGAGG | 58 |
| hsa-miR-520a-3p | AAAGUGCUUCCCUUUGGACUGU | 59 |
| hsa-miR-520g | ACAAAGUGCUUCCCUUUAGAGUGU | 60 |
| hsa-miR-521 | AACGCACUUCCCUUUAGAGUGU | 61 |
| hsa-miR-523 | GAACGCGCUUCCCUAUAGAGGGU | 62 |
| hsa-miR-548b-5p | AAAAGUAAUUGUGGUUUUGGCC | 63 |
| hsa-miR-548c-5p | AAAAGUAAUUGCGGUUUUUGCC | 64 |
| hsa-miR-548d-5p | AAAAGUAAUUGUGGUUUUUGCC | 65 |
| hsa-miR-590-3p | UAAUUUUAUGUAUAAGCUAGU | 66 |
| hsa-miR-593 | UGUCUCUGCUGGGGUUUCU | 67 |
| hsa-miR-597 | UGUGUCACUCGAUGACCACUGU | 68 |
| hsa-miR-628-3p | UCUAGUAAGAGUGGCAGUCGA | 69 |
| hsa-miR-650 | AGGAGGCAGCGCUCUCAGGAC | 70 |
| hsa-miR-657 | GGCAGGUUCUCACCCUCUCUAGG | 71 |
| hsa-miR-659 | CUUGGUUCAGGGAGGGUCCCCA | 72 |
| hsa-miR-668 | UGUCACUCGGCUCGGCCCACUAC | 73 |
| hsa-miR-720 | UCUCGCUGGGGCCUCCA | 74 |
| hsa-miR-874 | CUGCCCUGGCCCGAGGGACCGA | 75 |
| hsa-miR-886-3p | CGCGGGUGCUUACUGACCCUU | 76 |
| hsa-miR-942 | UCUUCUCUGUUUUGGCCAUGUG | 77 |

The invention relates more specifically to a method for diagnosis of muscular dystrophy or for evaluating the risk of developing or presenting a muscular dystrophy in a subject, comprising measuring the expression level of at least one miRNA in a sample of body fluid (for example a urine sample) of said subject. The invention can notably comprise comparing said expression level measured in said sample with a level obtained in a healthy reference sample, a difference between the expression level relative to the reference level being indicative of a muscular dystrophy in the subject.

The invention also relates to a method for diagnosis of muscular dystrophy or for evaluating the risk of developing or presenting a muscular dystrophy, comprising determination of the presence or of the expression level of at least one miRNA selected from the group consisting of the miRNAs listed in Table 1, in a sample of body fluid (for example a urine sample) of a subject.

The present invention notably relates to a method for diagnosis of muscular dystrophy, in particular Duchenne muscular dystrophy, comprising comparing:

a) the expression level of at least one miRNA in a sample of body fluid (e.g. of urine) of a subject (test sample), the miRNA being selected from the group consisting of the miRNAs in Table 1, and

b) the expression level of said miRNA in a healthy reference sample,

a statistically significant difference between the expression level in the test sample relative to the reference sample being indicative of a muscular dystrophy in the subject.

According to a particular aspect, the invention relates to a method for diagnosis of muscular dystrophy comprising the following steps:
measuring the expression level of at least one miRNA selected from the miRNAs in Table 1, in a sample of body fluid (especially urine) obtained from a subject to be tested (for example a subject suspected of having a muscular dystrophy); and
comparison:
   between the expression level of at least one of said miRNAs in said sample and the expression level of said miRNA in a healthy reference sample, or
   between the expression level of at least a first one of said miRNAs in the sample obtained from a patient suspected of having a muscular dystrophy and the expression level of said miRNA in a reference sample obtained from a patient with muscular dystrophy, especially DMD.

Thus, the expression levels of the miRNAs can be compared between a sample obtained from a patient suspected of having muscular dystrophy and a healthy reference sample, or a reference sample obtained from a patient with muscular dystrophy.

The existence, in the urine, of miRNAs specific to a muscular dystrophy, and in particular to DMD, has never been reported in earlier published works. Moreover, the following miRNAs have never been reported as being present in a body fluid and as being indicative of a muscular dystrophy: let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p. Thus, according to a particular embodiment, the method according to the invention comprises measuring the level of at least one miRNA selected from the group consisting of the miRNAs listed in the preceding sentence.

According to another particular embodiment, the method according to the invention comprises measuring the level, in a subject's urine sample, of at least one miRNA selected from the group consisting of let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p, measurement of a difference in the level of said miRNA in the subject's sample relative to the healthy reference sample being indicative of a possible muscular dystrophy.

The invention also relates to a method of monitoring the evolution of a muscular dystrophy, and a method for evaluating the efficacy of a therapeutic treatment of a muscular dystrophy. In this case, the method comprises measuring the expression level of at least one of the miRNAs mentioned above in a second sample of body fluid (notably urine) of a subject, this level in the subject's sample being compared with the level of said miRNA in a first reference sample that corresponds to a sample taken previously from the same subject. When monitoring the efficacy of a treatment, the first sample can have been taken before administering the therapeutic treatment to the subject, and the second sample can have been taken after administering the therapeutic treatment (for example several days/weeks/months after administration of the therapeutic treatment). Alternatively, the first and second samples can both be taken after administration of the therapeutic treatment (for example, the first sample is taken after treatment, on the same day as this treatment, or several days/weeks/months after the treatment, and the second sample is taken several days/weeks/months after the first sample).

The invention further relates to a kit and a multiwell support to be used for the diagnosis of muscular dystrophy.

### DETAILED DESCRIPTION OF THE INVENTION

The “microRNAs” (or miRNAs) are noncoding single-stranded RNAs with a length of about 17 to 26 nucleotides, which regulate gene expression by repressing translation of their target mRNA. The miRNAs that have been identified are recorded in the miRBase database, 14th version (http://microarn.saner.ac.uk).

In the context of the invention, a “reference sample”, when there is mention of a “healthy sample”, corresponds to a sample obtained from one or more subjects, preferably two or more, who do not have muscular dystrophy. The reference sample can also correspond to a sample obtained from one or more patients with muscular dystrophy. The reference expression levels can be determined by measuring the expression level of the miRNAs to be explored in one or more subjects. These reference levels can also be adjusted as a function of populations of specific subjects. In a preferred embodiment, the reference sample is obtained from a pool of healthy subjects. The expression profile of the miRNAs in the reference sample can preferably be generated starting from a population of two or more than two subjects. For example, the population can comprise 2, 4, 5, 10, 15, 20, 30, 40, 50 subjects, or more. In the context of methods for monitoring a muscular dystrophy or for monitoring the efficacy of a treatment, the reference sample is a sample taken from the subject who is to be monitored, but before monitoring has started.

“Muscular dystrophy” notably denotes Duchenne muscular dystrophy, Becker muscular dystrophy, the limb-girdle muscular dystrophies such as alpha- and gamma-sarcoglycanopathies. The invention relates more particularly to the investigation of Duchenne muscular dystrophy or Becker muscular dystrophy, more particularly Duchenne.

The term “body fluid” or “biological fluid” refers to the body fluid of a subject, notably of a human subject, i.e. any fluid taken from a subject, such as serum, plasma, whole blood, urine, cerebrospinal fluid or else saliva. According to a preferred embodiment, the body fluid used in the present invention is a urine sample.

“Subject” means a mammal, human or nonhuman, preferably human. The subject can have a predisposition to muscular dystrophy (revealed for example by genetic analysis, or a suspicion based on family history) or may have a diagnosed muscular dystrophy. The invention can also be applied in screening, when the subject does not have any symptom or known predisposition. In particular, the method according to the invention can be applied for mass screening in young children before the classical age when symptoms appear (0-5 years). The invention can also be applied for monitoring animal models of the disease, notably of dog or mouse models and more particularly in dogs: GRMD (Golden Retriever Muscular Dystrophy), LMD (Labrador Muscular Dystrophy) or CXMDj (Canine X-linked Muscular Dystrophy in Japan), during the preclinical development of treatments.

The term “expression level” of an miRNA in a sample corresponds to a measured value characteristic of an miRNA, but expressed either in arbitrary units, or in units of mass, of molecules or of concentration, or as a value normalized relative to another measurement, in particular as a value normalized relative to the amounts of the same miRNA in a reference sample (healthy or from a patient with muscular dystrophy).

The expression level of the miRNAs can be measured by any conventional method, such as
hybridization on “DNA chips”,
methods of sequencing with high throughput of a large number of individualized miRNAs,
real-time or digital quantitative PCR,
Northern blot,
or by any other method specific to miRNAs.

The expression level of the miRNAs can be measured by the “DNA chip” technique. The “DNA chip” technique is well known by a person skilled in the art. It involves hybridization of extracted miRNAs on a solid support composed of a nylon membrane, a silicon or glass surface, optionally nanobeads or particles, bearing oligonucleotides of known sequences fixed on the support or adhering to the latter. Complementarity of the fixed oligonucleotides with the sequences of the microRNAs or of their conversion products (amplification products, cDNA, RNA or cRNA) allows a signal to be generated (fluorescence, luminescence, radioactivity, electrical signal etc.) depending on the labeling techniques employed, at the level of the oligonucleotides immobilized on the supports (DNA chips). This signal is detected by special equipment and a value of intensity of this signal characteristic of each miRNA is thus recorded. Several types of chips intended for detection of miRNAs are already marketed, for example the GeneChip® miRNAs marketed by Affymetrix, miRcury arrays by Exiqon, miRXplore microarrays by Miltenyi.

In the case of high throughput analysis by sequencing, the miRNAs are extracted and purified from a sample, and isolated from one another by methods offered by the suppliers of sequencing equipment such as Roche, Invitrogen. This type of analysis consists of individualizing the molecules of the different microRNAs, carrying out an amplification step and sequencing the products (“clones of nucleic acids”) thus generated. By carrying out numerous sequences for identifying each of these “clones” (several thousand), it is possible to generate a list of the microRNAs present in a sample and quantify each of these miRNAs quite simply by counting how many times each sequence is found in the detailed list.

In a preferred embodiment of the invention, the miRNA assays are performed by quantitative PCR (real-time PCR or digital PCR). Real-time PCR makes it possible to obtain values, called Ct, corresponding to the number of cycles starting from which the fluorescence emitted exceeds a certain threshold, the threshold being fixed by the user at the start of the exponential phase. This Ct value is proportional to the quantity of cDNA (resulting from reverse transcription of the miRNAs to cDNA by reverse transcriptase) initially present in the sample. In the absence of a standard range specific to each cDNA, only relative quantification between samples is possible. Firstly, so as to be able to compare the contingent of each miRNA taken individually and present in the samples, the assay values for each miRNA are normalized with the data obtained for a noncoding RNA. It is also possible to normalize the expression of an miRNA relative to the average Ct of all the miRNAs of a PCR plate (384-well TLDA plates, comprising a different miRNA detected per well—cf. the examples for further details). Thus, the results can be normalized relative to several reference miRNAs whose abundance shows little variation in the urine. Digital PCR makes it possible to determine, from a starting sample, the exact number of copies of an miRNA that it contains, either following dilution of the PCR reaction in a large number of microwells (digital PCR technology, Life Technologies or Roche) or following dispersion of the PCR reaction in microdroplets of oil (Droplet technology, Bio-Rad).

The relative quantification of an miRNA between 2 types of samples is then obtained for example using the software SDS2.3, RQ manager (Applied Biosystems), and by the delta delta Ct method on Microsoft Excel spreadsheet or any other software for complex calculation.

When the expression levels of the miRNAs are analyzed by hybridization on “DNA chips”, or by Northern blot, or by sequencing, they can be expressed by formula I:

Quantity of miRNAx=intensity of the detection signal for miRNAx (I)

where “signal intensity” signifies quantity of fluorescence, of radioactivity or of luminescence recorded on the “DNA chips” by the appropriate detector, or number of identical sequences detected by the high-throughput analysis by sequencing. The quantities are expressed in arbitrary units.

The quantities of miRNA can be normalized relative to another assay, notably an RNA (RNAₙₒᵣₘ) whose concentration does not vary in the different types of samples analyzed. This normalization makes it possible to guarantee that we are comparing the expression levels of the miRNAs detectable in extracts whose RNA concentrations are similar between these various purified extracts. In this case, the normalized expression level for an miRNA in a sample is expressed by formula II:

Quantity of normalized miRNAx=intensity of the detection signal for miRNAx/signal intensity for RNAₙₒᵣₘ (II)

When the expression levels of the miRNAs are analyzed by real-time PCR, they can be expressed by formula III, which defines the quantity of miRNA present in the assay reaction mixture when the number of amplification cycles is equal to Ct (Quantity of miRNAx at Ct):

Quantity of miRNAx at Ct=Quantity of miRNA at t0×Efficacy^{-Ct}. (III)

where “Quantity of miRNA at t0” denotes the quantity of miRNA, or its equivalent in cDNA, at the moment when the assay reaction by PCR amplification is initiated. The expression “efficacy” in formula (III) signifies the value of the efficacy of PCR (fixed arbitrarily at 2 in the case of calculations by the delta delta Ct method). This value depends on various experimental parameters, and notably on the real-time PCR apparatus employed. Once this value has been measured for a particular protocol and a PCR machine has been configured, it is no longer necessary to measure this value each time before the calculation, unless the experimental protocol and/or the operating conditions of the machine have been changed for the given experiment.

In a particular embodiment, the expression level of the miRNAs is assayed by real-time quantitative PCR.

Tables 2 and 3 below describe the expression profile of miRNAs whose expression is altered in DMD patients, relative to the expression profile observed in healthy subjects. The inventors were able to demonstrate a difference in expression between the patients according to their age, thus the information is classified according to this criterion.

TABLE 2
expression profile of the indicative miRNAs in patients/subjects aged 3-8 years

| | Expression in the patients relative to |
|---|---|
| urinary miRNA | healthy subjects |
| let-7a | Increase |
| let-7b | Increase |
| let-7c | Increase |
| let-7d | Increase |
| let-7e | Increase |
| let-7f | Increase |
| let-7g | Increase |
| miR-151-5p | Increase |
| miR-15a | Increase |
| miR-15b | Increase |
| miR-182 | Increase |
| miR-183 | Increase |
| miR-192* | Increase |
| miR-196b | Increase |
| miR-200b* | Increase |
| miR-206 | Increase |
| miR-224 | Increase |
| miR-23b | Increase |
| miR-26b | Increase |
| miR-28-5p | Increase |
| miR-30d | Increase |
| miR-30e-3p | Increase |
| miR-335 | Increase |
| miR-33a* | Increase |
| miR-487b | Increase |
| miR-490-3p | Increase |
| miR-492 | Increase |
| miR-502-3p | Increase |
| miR-505* | Increase |
| miR-520a-3p | Increase |
| miR-548d-5p | Increase |
| miR-590-3p | Increase |
| miR-628-3p | Increase |
| miR-659 | Increase |
| miR-942 | Increase |
| miR-1244 | Decrease |
| miR-328 | Decrease |
| miR-484 | Decrease |
| miR-494 | Decrease |
| miR-593 | Decrease |
| miR-650 | Decrease |
| miR-657 | Decrease |
| miR-668 | Decrease |
| miR-720 | Decrease |
| miR-886-3p | Decrease |

TABLE 3
expression profile of the indicative miRNAs in
patients/subjects aged 13-18 years

| | Expression in the patients relative to |
|---|---|
| urinary miRNA | healthy subjects |
| miR-183 | Increase |
| miR-193a-3p | Increase |
| miR-198 | Increase |
| miR-208 | Increase |
| miR-214 | Increase |
| miR-220 | Increase |
| miR-328 | Increase |
| miR-346 | Increase |
| miR-34c-5p | Increase |
| miR-373 | Increase |
| miR-381 | Increase |
| miR-410 | Increase |
| miR-433 | Increase |
| miR-490-3p | Increase |
| miR-493 | Increase |
| miR-494 | Increase |
| miR-511 | Increase |
| miR-517b | Increase |
| miR-518a-3p | Increase |
| miR-518b | Increase |
| miR-518e | Increase |
| miR-518f | Increase |
| miR-520a-3p | Increase |
| miR-520g | Increase |
| miR-521 | Increase |
| miR-523 | Increase |
| miR-548b-5p | Increase |
| miR-548c-5p | Increase |
| miR-548d-5p | Increase |
| miR-597 | Increase |
| let-7b | Decrease |
| let-7c | Decrease |
| let-7e | Decrease |
| let-7f | Decrease |
| miR-139-5p | Decrease |
| miR-155 | Decrease |
| miR-15a | Decrease |
| miR-216b | Decrease |
| miR-23a | Decrease |
| miR-376c | Decrease |
| miR-412 | Decrease |
| miR-423-5p | Decrease |
| miR-492 | Decrease |
| miR-502-3p | Decrease |
| miR-874 | Decrease |

Legend of Tables 2 and 3 increase: higher expression in the patients relative to the healthy subjects; decrease: lower expression in the patients relative to the healthy subjects.

All of the miRNAs listed above vary in the samples from patients relative to the healthy subjects. The invention therefore relates to a method (a) for diagnosis of a muscular dystrophy, (b) for monitoring the evolution of a muscular dystrophy, and (c) for evaluating the efficacy of a therapeutic treatment of a muscular dystrophy, comprising determination of a change in expression level of one or more of these miRNAs in a sample of body fluid from a subject relative to the expression level in a reference sample.

In a particular embodiment, a first category of miRNAs corresponds to those that are over-represented in the urine of DMD patients (denoted by the category “increase” in Tables 2 and 3). If one or more miRNAs of this first category are used in a method according to the invention:
higher expression in the test subject relative to a reference obtained in a sample from a healthy subject will be indicative of a muscular dystrophy (method of diagnosis);
higher expression in a sample from the test subject taken at a time T2 relative to a sample from the same test subject taken at a time T1 (T1 preceding T2 chronologically) will be indicative of progression of the disease (method of prognosis, or method for monitoring a muscular dystrophy);
in the context of a treatment of a muscular dystrophy in a patient, lower expression in a sample from the test subject taken at a time T2 relative to a sample from the same test subject taken at a time T1 (T1 preceding T2 chronologically) will be indicative of effective treatment of the disease (method of monitoring the efficacy of a treatment of a muscular dystrophy).

A second category of miRNAs is under-represented in the urine of DMD patients, relative to healthy subjects (denoted in the category “decrease” in Tables 2 and 3). If one or more miRNAs of this second category are used in a method according to the invention:
lower expression in the test subject relative to a reference obtained in a sample from a healthy subject will be indicative of a muscular dystrophy (method of diagnosis);
lower expression in a sample from the test subject taken at a time T2 relative to a sample from the same test subject taken at a time T1 (T1 preceding T2 chronologically) will be indicative of progression of the disease (method of prognosis, or method for monitoring a muscular dystrophy);
in the context of a treatment of a muscular dystrophy in a patient, higher expression in a sample from the test subject taken at a time T2 relative to a sample from the same test subject taken at a time T1 (T1 preceding T2 chronologically) will be indicative of effective treatment of the disease (method for monitoring the efficacy of a treatment of a muscular dystrophy).

“Higher expression level” or “lower expression level” means an expression level whose change is statistically significant, according to procedures well known by a person skilled in the art.

The above description of the two categories of miRNAs identified and exploitation of them in a method of diagnosis according to the invention employs a reference sample obtained from a healthy subject. Of course, the changes in expression investigated will be reversed when the reference sample is from a patient with muscular dystrophy.

The methods according to the invention notably comprise detection of at least one miRNA selected from the group consisting of the miRNAs in Tables 2 and 3.

According to a first embodiment variant, the miRNAs detected are selected from the miRNAs in Table 4.

TABLE 4

| | | | |
|---|---|---|---|
| let-7f | miR-23b | miR-193a-3p | miR-518f |
| let-7a | miR-492 | miR-381 | miR-886-3p |
| miR-548d-5p | let-7c | miR-34c-5p | miR-650 |
| miR-183 | let-7e | miR-628-3p | miR-720 |
| miR-490-3p | miR-15b | miR-659 | miR-593 |
| miR-520a-3p | miR-487b | miR-505* | miR-657 |
| miR-590-3p | miR-410 | let-7b | miR-502-3p |
| miR-15a | miR-139-5p | let-7d | miR-198 |
| miR-1244 | miR-216b | let-7g | miR-214 |
| miR-328 | miR-423-5p | miR-196b | miR-220 |
| miR-494 | miR-484 | miR-26b | miR-373 |
| miR-668 | miR-23a | miR-942 | miR-511 |
| miR-208 | miR-376c | miR-200b* | miR-517b |
| miR-521 | miR-412 | miR-523 | miR-518a-3p |
| miR-597 | miR-433 | miR-346 | miR-518b |
| miR-874 | miR-206 | miR-155 | miR-518e |
| miR-224 | miR-335 | miR-548b-5p | miR-520g |
| miR-182 | miR-33a* | miR-548c-5p | miR-493 |

According to a second embodiment variant, the miRNAs detected are selected from the miRNAs in Table 5.

TABLE 5

| | | | |
|---|---|---|---|
| let-7f | miR-494 | let-7c | miR-376c |
| let-7a | miR-668 | let-7e | miR-412 |
| miR-548d-5p | miR-208 | miR-15b | miR-433 |
| miR-183 | miR-521 | miR-487b | miR-206 |
| miR-490-3p | miR-597 | miR-410 | miR-335 |
| miR-520a-3p | miR-874 | miR-139-5p | miR-33a* |
| miR-590-3p | miR-224 | miR-216b | miR-193a-3p |
| miR-15a | miR-182 | miR-423-5p | miR-381 |
| miR-1244 | miR-23b | miR-484 | miR-34c-5p |
| miR-328 | miR-492 | miR-23a | |

According to a third embodiment variant, the miRNAs detected are selected from the miRNAs in Table 6.

TABLE 6

| | | |
|---|---|---|
| let-7f | miR-590-3p | miR-208 |
| let-7a | miR-15a | miR-521 |
| miR-548d-5p | miR-1244 | miR-597 |
| miR-183 | miR-328 | miR-874 |
| miR-490-3p | miR-494 | |
| miR-520a-3p | miR-668 | |

According to a particular embodiment, the sample of body fluid, in particular a urine sample, is from a human subject and the miRNA or miRNAs detected are selected from the group consisting of the miRNAs in Tables 2 and 3, or from the miRNAs in Table 2 or 3 and also appear in one of Tables 4, 5 and 6.

The diagnosis (or the evaluation of risk), the prognosis or the efficacy of treatment can moreover be confirmed in procedures following the methods according to the invention, comprising known steps for evaluating a muscular dystrophy (for example, determination of the level of creatine kinase, searching for specific markers in muscle biopsies, genomic analysis, etc.). Thus, a particular embodiment of the method of diagnosis according to the invention as described above further comprises a step of confirming the diagnosis using an alternative method for evaluating a muscular dystrophy.

The invention also relates to a kit for diagnosis of a muscular dystrophy, said kit comprising means for detection or for assay of at least one miRNA selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p. According to a particular embodiment, the kit comprises the means for detection or for assay of all of the miRNAs on this list. According to a particular embodiment, the kit comprises means for detection or for assay of one or more miRNAs (especially all) selected from the miRNAs listed in Table 4, Table 5 or Table 6. According to a specific embodiment, the means for detection or for assay in the kit consist of means for detection or for assay of one or more of the miRNAs in Tables 2 and 3, or of one or more of the miRNAs listed in each of Tables 4, 5 and 6. According to a particular embodiment, the miRNAs detected or assayed using the kit consist of all of the miRNAs in Table 4, more particularly all of the miRNAs in Table 5, and even more particularly all of the miRNAs in Table 6.

As an illustration, the kit according to the invention can be a kit for carrying out real-time PCR and can also contain a reverse transcriptase, a DNA polymerase, one or more buffers suitable for the reactions to be employed, specific probes of the amplified regions (for example Taqman® probes), or specific markers of the double-stranded DNA such as SYBR Green.

The invention also relates to a set of nucleotide sequences, said set comprising primer pairs usable for specifically amplifying at least two miRNAs selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3 in a PCR experiment. According to a variant, the nucleotide sequences permit amplification of one or more miRNAs from each of Tables 4, 5 and 6. According to a particular embodiment, the set of nucleotide sequences comprises primer pairs permitting specific amplification of all of the miRNAs listed above. According to an embodiment variant, the set of nucleotide sequences can also comprise a nucleotide sequence usable as a labeled probe for detection and quantification of the amplified fragments (for example a probe usable in the TaqMan real-time PCR system).

The invention also relates to a set of nucleotide sequences comprising one or more labeled oligonucleotides usable for specific detection of at least two miRNAs in Table 1, for example in a Northern Blot experiment. In a particular embodiment, the set of sequences contains specific oligonucleotides of each of the miRNAs in Table 1, Table 2, Table 3, Table 4, Table 5 or Table 6.

The invention also relates to a multiwell support for PCR, comprising at least two PCR primer pairs each specific to a different miRNA from Table 1, Table 2, Table 3, Table 4, Table 5 or Table 6, each of the primer pairs being arranged in a different well of the support. According to a specific variant, the support contains primer pairs consisting of primers specific to at least two miRNAs in Table 1, 2, 3, 4, 5 or 6, each of the primer pairs being arranged in a different well of the support. According to a particular embodiment, the multiwell support comprises primer pairs specific to all of the miRNAs in Table 1, Table 2, Table 3, Table 4, Table 5 or Table 6, each of these primer pairs being arranged in a different well. According to another specific embodiment, the support contains primer pairs consisting of primers specific to all of the miRNAs in Table 1, 2, 3, 4, 5 or 6, each of the primer pairs being arranged in a different well of the support.

The present invention is illustrated by the following figures and examples.

### LEGEND OF THE FIGURES

FIG. 1: (top) number of different miRNAs detected per category of samples after expression profile by TLDA cards A and B (patients 3-8 years) or TLDA A (patients 13-18 years). (bottom) average Ct per sample category. Healthy subjects aged 3-8 years (5 samples), DMD 3-8 years (5 samples), healthy subjects aged 13-18 years (3 samples), DMD 13-18 years (2 samples).

FIG. 2: heatmaps comprising the abundances of each miRNA identified for each donor tested, and hierarchic grouping of the donors according to expression of the candidate miRNAs. (top) heatmap for those aged 3-8 years. (bottom) heatmap for those aged 13-18 years. The heatmaps and the calculations of hierarchic grouping are performed using the software CIMminer (http://discover.nci.nih.gov/cimminer/)

FIG. 3: example of deregulated miRNAs in the urine of DMD patients. The abundance of miRNAs is shown as a function of the group of patients.

### EXAMPLES

### Material and Methods

The urine is collected in sterile containers. In the next half-hour, it is centrifuged at 2000 rpm for 5 min in order to remove the cells that are present. The supernatant is then recovered, aliquoted and frozen at −80° C.

The investigation on card A is based on urine samples from 4 DMD patients and 6 healthy subjects aged from 3 to 8 years or on 2 DMD patients and 3 healthy subjects aged from 13 to 18 years.

The investigation on card B is based on urine samples from 4 DMD patients and 5 healthy subjects.

10 ml of urine is used for extracting the total RNAs containing the microRNAs using the kit “Urine total RNA maxi kit, slurry format” from Norgen Biotek, according to the supplier's protocol. The RNAs are eluted in 2 successive elutions of 1004. They are then precipitated overnight at −20° C. in the presence of sodium acetate, absolute ethanol and linear acrylamide (Ambion) according to the Ambion protocol. The RNAs are then resuspended in water without RNAse.

Quality control of the RNAs is then performed in 3 steps: 1) determination by absorbance at 260 nm (Nanodrop 8000, Thermo Scientific) 2) capillary electrophoresis on small and pico RNA chip (Agilent Technologies) 3) amplification of 3 small control urinary RNAs by RT-qPCR (miR-16, miR-377*, U6).

100 ng of total RNA is then submitted to multiplex reverse transcription (Megaplex pools, Applied biosystems). We perform 2 reverse transcriptions starting from 2 different primer pools: pools A and B. Together, they cover detection of 762 different microRNAs, which represents about half of the 1424 known human miRNAs (miRbase, www.mirbase.org, release 17 Apr. 2011). The complementary DNAs obtained then undergo a preamplification step (preAmp master mix and preAmp primer pools, Applied Biosystems) before being deposited on TLDA (Taqman Low Density Array) plates. This technology was developed by Applied Biosystems, and consists of the simultaneous detection of 381 miRNAs on a 384-well plate by RT-qPCR. The relative quantity of each miRNA is determined by normalizing with the average Ct (cycle threshold) of each sample (Mestdagh, Genome Biol 2009), and by using a sample from a healthy donor as reference (ddCt method). The ratios of the relative quantities of each miRNA between the population of healthy donors and the population of DMD patients are then determined.

The relative quantities are calculated by the delta delta Ct method. With dCt (miR)=Ct miR−Ct calibrator;

ddCt (miR)=dCt (reference)−dCt (miR); and

Relative quantity (miR)=2̂delta delta Ct (miR).

The reference corresponds to the mean value obtained for a given miR in the healthy donors.

The calibrator corresponds to the average Ct of the whole TLDA plate.

Results:

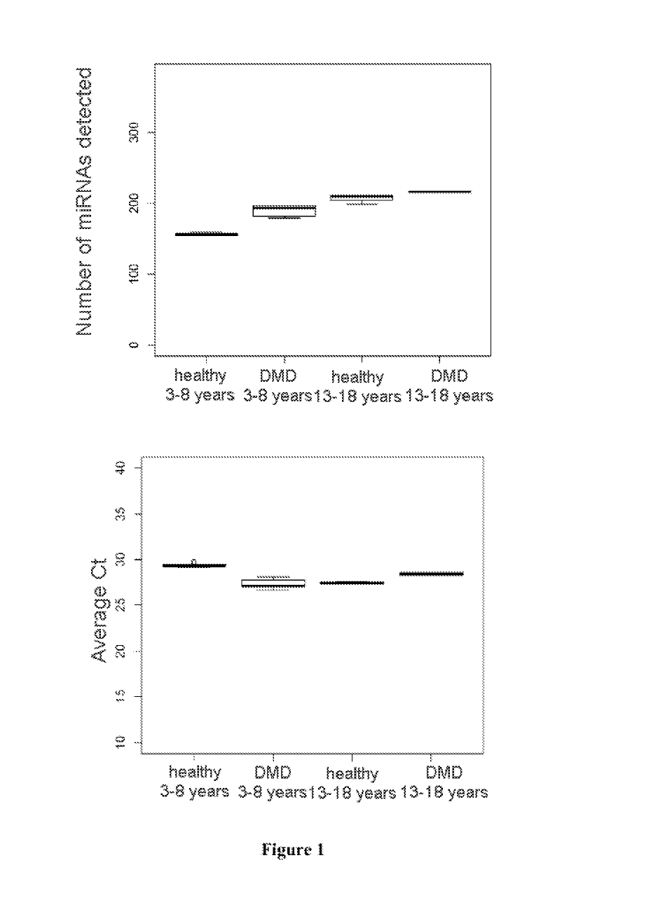

We only considered the miRNAs detected with a Ct below 35 for a threshold of 0.1, according to the RQ manager software (Applied Biosystems). Among the subjects aged 3-8 years, considering only panel A of the TLDA cards used, and for each urine sample, we detected an average of 172 different miRNAs, the average Ct of each sample being equal to 28.2 (FIG. 1). Among the subjects aged 13-18 years, considering only panel A of the TLDA cards used, and for each urine sample, we detected an average of 210 different miRNAs, the average Ct of each sample being equal to 27.8 (FIG. 1). Panel B was only tested for the donors aged 3-8 years and made it possible to detect an average of 160 additional miRNAs (i.e. about 330 different miRNAs detectable in the urine of the donors aged 3-8 years). We therefore observe a quite large abundance and variety of miRNAs in the urine.

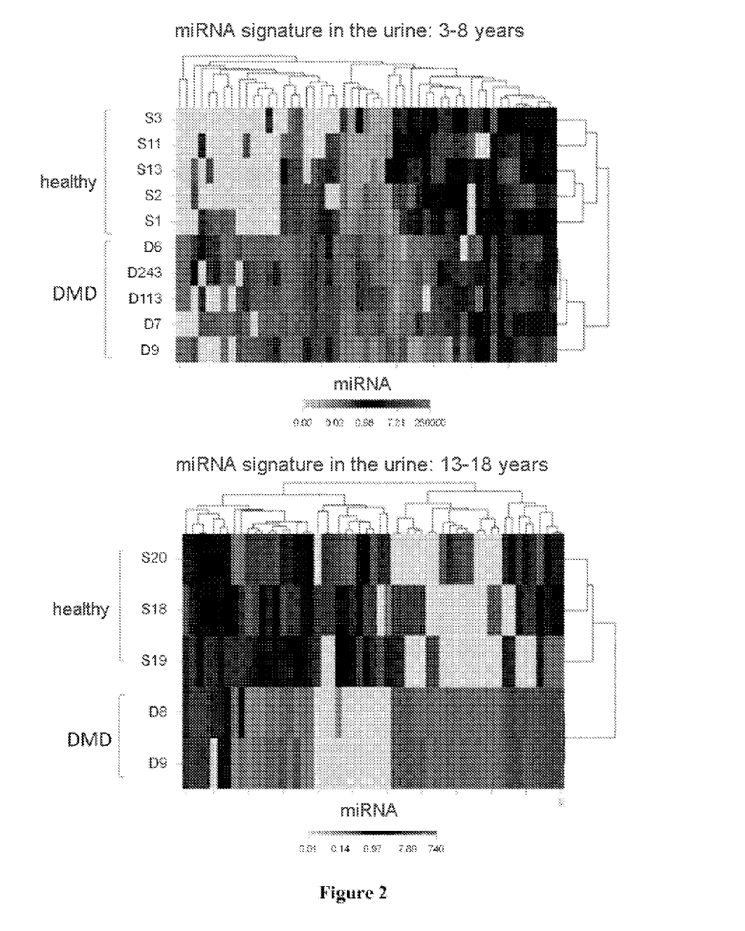

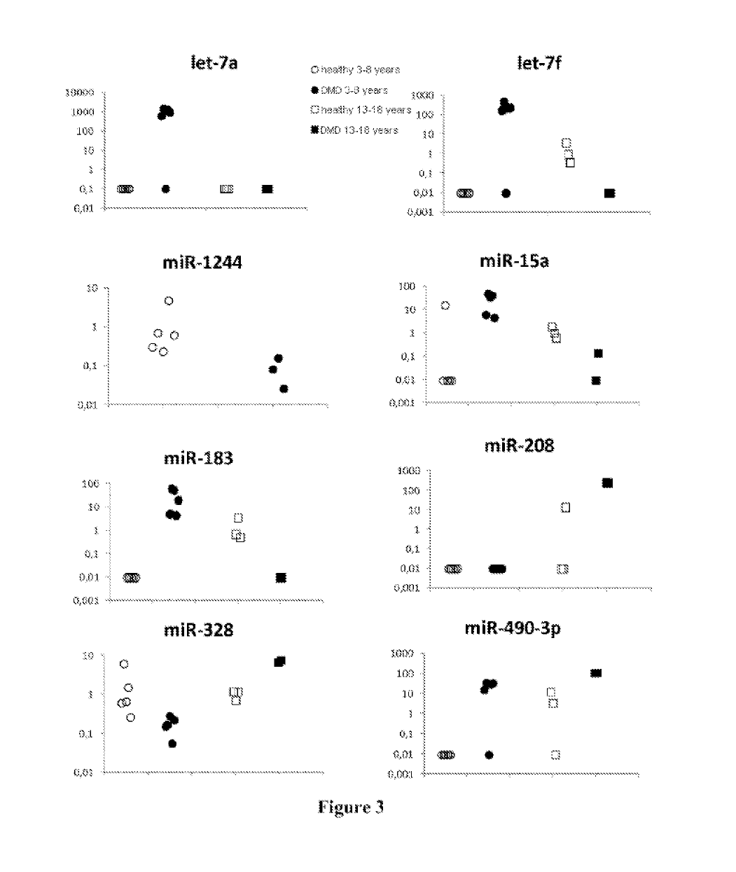

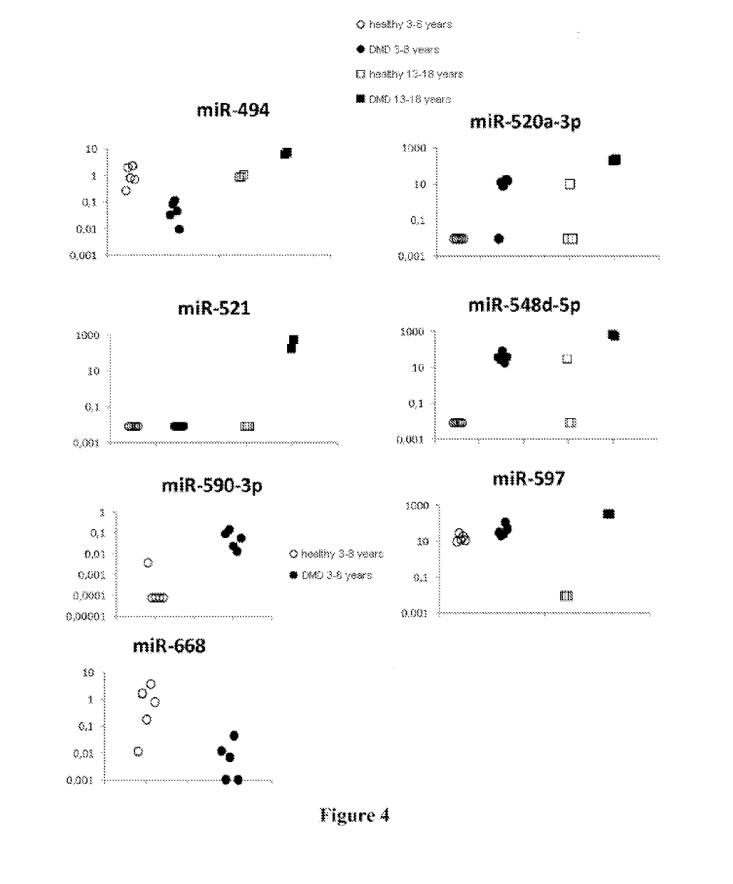

Finally we determined the list of miRNAs represented differently in the urine of the DMD patients relative to the healthy donors, by comparing the subjects of equivalent age (1: group 3-8 years; 2: group 13-18 years). The miRNAs are shown in Table 7, indicating for each miRNA its level of deregulation in the urine of the groups aged 3-8 years and 13-18 years (difference factor), its category (increased, decreased in the DMD patients relative to the healthy subjects), and its potential as biomarkers (score out of 4). The miRNAs with very high potential (potential=4) show large modification factors and/or deregulation in the 2 age groups. The miRNAs with good potential (potential=1) show small but significant difference factors. The miRNAs with high potential or with intermediate potential have a score of 3 or 2. FIG. 2 shows these results in the form of 2 heatmaps, one per age group. Based on the data for abundance of the different urinary miRNAs selected in Table 7, the hierarchic grouping algorithm used (http://discover.nci.nih.gov/cimminer/) allows effective separation of the donors as a function of their healthy or DMD status. Thus, this result shows that the expression of the miRNAs identified can be used as a signature of the DMD pathology. FIG. 3 gives examples of miRNAs deregulated in the DMD patients.

TABLE 7

| | Difference | | |
|---|---|---|---|
| | factor (DMD/ | | |
| | healthy) 3-8 | increases/decreases in | |
| urinary miRNA | years | DMD 3-8 years | potential/4 |
| hsa-let-7f | 80000 | increases | 4 |
| hsa-let-7a | 20000 | increases | 4 |
| hsa-miR-548d-5p | 4000 | increases | 4 |
| hsa-miR-183 | 2000 | increases | 4 |
| hsa-miR-490-3p | 2000 | increases | 4 |
| hsa-miR-520a-3p | 1000 | increases | 4 |
| hsa-miR-590-3p | 80 | increases | 4 |
| hsa-miR-15a | 9 | increases | 4 |
| hsa-miR-224 | 8000 | increases | 3 |
| hsa-miR-182 | 25 | increases | 3 |
| hsa-miR-23b | 8 | increases | 3 |
| hsa-miR-492 | 6 | increases | 3 |
| hsa-let-7c | 5 | increases | 3 |
| hsa-let-7e | 5 | increases | 3 |
| hsa-miR-15b | 5 | increases | 3 |
| hsa-miR-206 | 3 | increases | 3 |
| hsa-miR-335 | 3 | increases | 3 |
| hsa-miR-33a* | 3 | increases | 3 |
| hsa-miR-487b | 3 | increases | 3 |
| hsa-miR-628-3p | 12 | increases | 2 |
| hsa-miR-659 | 9 | increases | 2 |
| hsa-miR-505* | 7 | increases | 2 |
| hsa-let-7b | 5 | increases | 2 |
| hsa-let-7d | 5 | increases | 2 |
| hsa-let-7g | 5 | increases | 2 |
| hsa-miR-196b | 5 | increases | 2 |
| hsa-miR-26b | 5 | increases | 2 |
| hsa-miR-942 | 5 | increases | 2 |
| hsa-miR-200b* | 3 | increases | 2 |
| hsa-miR-502-3p | 3 | increases | 2 |
| hsa-miR-151-5p | 3 | increases | 1 |
| hsa-miR-192* | 3 | increases | 1 |
| hsa-miR-28-5p | 3 | increases | 1 |
| hsa-miR-30d | 3 | increases | 1 |
| hsa-miR-30e-3p | 3 | increases | 1 |
| hsa-miR-668 | −100 | decreases | 4 |
| hsa-miR-1244 | −20 | decreases | 4 |
| hsa-miR-494 | −20 | decreases | 4 |
| hsa-miR-328 | −10 | decreases | 4 |
| hsa-miR-484 | −5 | decreases | 3 |
| hsa-miR-657 | −17 | decreases | 2 |
| hsa-miR-593 | −13 | decreases | 2 |
| hsa-miR-650 | −12 | decreases | 2 |
| hsa-miR-720 | −12 | decreases | 2 |
| hsa-miR-886-3p | −8 | decreases | 2 |
| | Difference | | |
| | factor (DMD/ | | |
| | healthy) 13-18 | increases/decreases in | potential/ |
| urinary miRNA | years | DMD 13-18 years | 4 |
| hsa-miR-521 | 40000 | increases | 4 |
| hsa-miR-597 | 30000 | increases | 4 |
| hsa-miR-520a-3p | 67 | increases | 4 |
| hsa-miR-548d-5p | 63 | increases | 4 |
| hsa-miR-208 | 54 | increases | 4 |
| hsa-miR-490-3p | 20 | increases | 4 |
| hsa-miR-328 | 7 | increases | 4 |
| hsa-miR-494 | 7 | increases | 4 |
| hsa-miR-193a-3p | 35 | increases | 3 |
| hsa-miR-34c-5p | 26 | increases | 3 |
| hsa-miR-433 | 20 | increases | 3 |
| hsa-miR-381 | 15 | increases | 3 |
| hsa-miR-410 | 14 | increases | 3 |
| hsa-miR-518a-3p | 173 | increases | 2 |
| hsa-miR-198 | 84 | increases | 2 |
| hsa-miR-511 | 58 | increases | 2 |
| hsa-miR-373 | 36 | increases | 2 |
| hsa-miR-548c-5p | 29 | increases | 2 |
| hsa-miR-220 | 26 | increases | 2 |
| hsa-miR-346 | 24 | increases | 2 |
| hsa-miR-518b | 24 | increases | 2 |
| hsa-miR-214 | 22 | increases | 2 |
| hsa-miR-520g | 22 | increases | 2 |
| hsa-miR-548b-5p | 21 | increases | 2 |
| hsa-miR-517b | 17 | increases | 2 |
| hsa-miR-518e | 17 | increases | 2 |
| hsa-miR-518f | 17 | increases | 2 |
| hsa-miR-493 | 13 | increases | 2 |
| hsa-miR-523 | 12 | increases | 2 |
| hsa-miR-874 | −2000 | decreases | 4 |
| hsa-let-7f | −171 | decreases | 4 |
| hsa-miR-183 | −162 | decreases | 4 |
| hsa-miR-15a | −16 | decreases | 4 |
| hsa-miR-412 | −620 | decreases | 3 |
| hsa-miR-216b | −533 | decreases | 3 |
| hsa-miR-23a | −464 | decreases | 3 |
| hsa-miR-139-5p | −216 | decreases | 3 |
| hsa-miR-376c | −128 | decreases | 3 |
| hsa-miR-492 | −123 | decreases | 3 |
| hsa-miR-423-5p | −120 | decreases | 3 |
| hsa-let-7e | −11 | decreases | 3 |
| hsa-let-7c | −10 | decreases | 3 |
| has-miR-155 | −17 | decreases | 2 |
| hsa-let-7b | −10 | decreases | 2 |

### REFERENCES

Batchelor, C. L. and S. J. Winder (2006). “Sparks, signals and shock absorbers: how dystrophin loss causes muscular dystrophy.” Trends Cell Biol 16(4): 198-205.

Bushby, K., R. Finkel, et al. “Diagnosis and management of Duchenne muscular dystrophy, part 1: diagnosis, and pharmacological and psychosocial management.” Lancet Neurol 9(1): 77-93.

Cacchiarelli, D., I. Legnini, et al. “miRNAs as serum biomarkers for Duchenne muscular dystrophy.” EMBO Mol Med 3(5): 258-65.

Cacchiarelli, D., J. Martone, et al. “MicroRNAs involved in molecular circuitries relevant for the Duchenne muscular dystrophy pathogenesis are controlled by the dystrophin/nNOS pathway.” Cell Metab 12(4): 341-51.

Cirak, S., V. Arechavala-Gomeza, et al. “Exon skipping and dystrophin restoration in patients with Duchenne muscular dystrophy after systemic phosphorodiamidate morpholino oligomer treatment: an open-label, phase 2, dose-escalation study.” Lancet.

Gidlof, O., P. Andersson, et al. “Cardiospecific microRNA Plasma Levels Correlate with Troponin and Cardiac Function in Patients with ST Elevation Myocardial Infarction, Are Selectively Dependent on Renal Elimination, and Can Be Detected in Urine Samples.” Cardiology 118(4): 217-226.

Greco, S., M. De Simone, et al. (2009). “Common micro-RNA signature in skeletal muscle damage and regeneration induced by Duchenne muscular dystrophy and acute ischemia.” Faseb J 23(10): 3335-46.

Hanke, M., K. Hoefig, et al. “A robust methodology to study urine microRNA as tumor marker: microRNA-126 and microRNA-182 are related to urinary bladder cancer.” Urol Oncol 28(6): 655-61.

Le Rumeur, E., S. J. Winder, et al. “Dystrophin: more than just the sum of its parts.” Biochim Biophys Acta 1804(9): 1713-22.

Lott, J. A. and P. W. Landesman (1984). “The enzymology of skeletal muscle disorders.” Crit Rev Clin Lab Sci 20(2): 153-90.

Lu, Q. L., T. Yokota, et al. “The status of exon skipping as a therapeutic approach to duchenne muscular dystrophy.” Mol Ther 19(1): 9-15.

Muntoni, F., S. Torelli, et al. (2003). “Dystrophin and mutations: one gene, several proteins, multiple phenotypes.” Lancet Neurol 2(12): 731-40.

Nicholson, G. A., G. J. Morgan, et al. (1986). “The effect of aerobic exercise on serum creatine kinase activities.” Muscle Nerve 9(9): 820-4.

Wang, G., L. S. Tam, et al. “Serum and urinary free microRNA level in patients with systemic lupus erythematosus.” Lupus 20(5): 493-500.

Weber, J. A., D. H. Baxter, et al. “The microRNA spectrum in 12 body fluids.” Clin Chem 56(11): 1733-41.

Yamada, Y., H. Enokida, et al. “MiR-96 and miR-183 detection in urine serve as potential tumor markers of urothelial carcinoma: correlation with stage and grade, and comparison with urinary cytology.” Cancer Sci 102(3): 522-9.

1-15. (canceled)

16. A method for diagnosis or for evaluating the risk of developing or presenting a muscular dystrophy in a subject, comprising measuring the expression level of at least one microRNA in a urine sample of said subject and comparing said expression level measured in said urine sample with a level obtained in a healthy reference sample, a difference between the expression level relative to the reference sample being indicative of a dystrophy in the subject.

17. The method according to claim 16, in which the at least one microRNA is selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p.

18. The method according to claim 17, the method comprising measuring all of the miRNAs listed.

19. The method according to claim 16, said microRNA or said microRNAs being selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p, miR-198, miR-214, miR-220, miR-373, miR-511, miR-517b, miR-518a-3p, miR-518b, miR-518e, miR-520g and miR-493.

20. The method according to claim 16, said microRNA (miRNA) or said microRNAs (miRNAs) being selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381 and miR-34c-5p.

21. The method according to claim 16, said miRNA or said miRNAs being selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597 and miR-874.

22. The method according to claim 16, wherein said muscular dystrophy is Duchenne muscular dystrophy or Becker muscular dystrophy.

23. A method for diagnosis or for evaluating the risk of developing or presenting a muscular dystrophy in a subject, comprising measuring the expression level, in a sample of body fluid of said subject, of at least one microRNA selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p.

24. The method according to claim 23, the sample being a urine sample.

25. A method for monitoring the evolution of a muscular dystrophy comprising measuring the expression level of at least one microRNA selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p in a second sample of body fluid of a subject, this level in the subject's sample being compared with the level of said microRNA in a first reference sample that corresponds to a sample taken previously from the same subject;
the evolution of the expression level of the microRNA or microRNAs selected being indicative of progression of the muscular dystrophy.

26. The method according to claim 25, the sample being a urine sample.

27. A method for determining the efficacy of a therapeutic treatment of a muscular dystrophy in a subject, comprising
a) measuring the expression level of one or more microRNAs in a body fluid of said subject, whereby a reference level is determined; then
b) measuring the expression level of said at least one microRNA selected in step a) in a second sample of biological fluid taken from the same subject at a given time after administration of the therapeutic treatment, whereby a test level is determined; and
c) comparing the control and test levels, the evolution of the expression level of the microRNAs selected being indicative of an effective treatment of the subject;
said miRNA or said miRNAs being selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p.

28. The method according to claim 27, the sample being a urine sample.

29. A method for (a) monitoring the evolution of a muscular dystrophy or (b) determining the efficacy of a therapeutic treatment of a muscular dystrophy in a subject, comprising measuring the expression level, in a urine sample of said subject, of at least one microRNA selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p.

30. A kit comprising means for detection or for assay of microRNAs, the means for detection or for assay in the kit consisting of means for detection or for assay of one or more miRNAs selected from let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p.

31. The kit according to claim 30, each of the miRNAs being detected or assayed by means of a probe and/or specific primer pair.

32. A multiwell support for PCR comprising PCR primer pairs consisting of specific primers of at least two miRNAs selected from the group consisting of let-7f, let-7a, miR-548d-5p, miR-183, miR-490-3p, miR-520a-3p, miR-590-3p, miR-15a, miR-1244, miR-328, miR-494, miR-668, miR-208, miR-521, miR-597, miR-874, miR-224, miR-182, miR-23b, miR-492, let-7c, let-7e, miR-15b, miR-487b, miR-410, miR-139-5p, miR-216b, miR-423-5p, miR-484, miR-23a, miR-376c, miR-412, miR-433, miR-206, miR-335, miR-33a*, miR-193a-3p, miR-381, miR-34c-5p, miR-628-3p, miR-659, miR-505*, let-7b, let-7d, let-7g, miR-196b, miR-26b, miR-942, miR-200b*, miR-523, miR-346, miR-155, miR-548b-5p, miR-548c-5p, miR-518f, miR-886-3p, miR-650, miR-720, miR-593, miR-657, miR-502-3p miR-198, miR-214, miR-220, miR-373, miR-511, miR-517b, miR-518a-3p, miR-518b, miR-518e, miR-520g, miR-493, miR-151-5p, miR-192*, miR-28-5p, miR-30d and miR-30e-3p, each of the primer pairs being arranged in a different well of the support.


SEQUENCE LISTING (77 sequences)

SEQ ID NO: 1 - RNA, 22 nt, homo sapiens
ugagguaguagguuguauaguu

SEQ ID NO: 2 - RNA, 22 nt, homo sapiens
ugagguaguagguugugugguu

SEQ ID NO: 3 - RNA, 22 nt, homo sapiens
ugagguaguagguuguaugguu

SEQ ID NO: 4 - RNA, 22 nt, homo sapiens
agagguaguagguugcauaguu

SEQ ID NO: 5 - RNA, 22 nt, homo sapiens
ugagguaggagguuguauaguu

SEQ ID NO: 6 - RNA, 22 nt, homo sapiens
ugagguaguagauuguauaguu

SEQ ID NO: 7 - RNA, 22 nt, homo sapiens
ugagguaguaguuuguacaguu

SEQ ID NO: 8 - RNA, 26 nt, homo sapiens
aaguaguugguuuguaugagaugguu

SEQ ID NO: 9 - RNA, 23 nt, homo sapiens
ucuacagugcacgugucuccagu

SEQ ID NO: 10 - RNA, 21 nt, homo sapiens
ucgaggagcucacagucuagu

SEQ ID NO: 11 - RNA, 23 nt, homo sapiens
uuaaugcuaaucgugauaggggu

SEQ ID NO: 12 - RNA, 22 nt, homo sapiens
uagcagcacauaaugguuugug

SEQ ID NO: 13 - RNA, 22 nt, homo sapiens
uagcagcacaucaugguuuaca

SEQ ID NO: 14 - RNA, 24 nt, homo sapiens
uuuggcaaugguagaacucacacu

SEQ ID NO: 15 - RNA, 22 nt, homo sapiens
uauggcacugguagaauucacu

SEQ ID NO: 16 - RNA, 22 nt, homo sapiens
cugccaauuccauaggucacag

SEQ ID NO: 17 - RNA, 22 nt, homo sapiens
aacuggccuacaaagucccagu

SEQ ID NO: 18 - RNA, 22 nt, homo sapiens
uagguaguuuccuguuguuggg

SEQ ID NO: 19 - RNA, 22 nt, homo sapiens
gguccagaggggagauagguuc

SEQ ID NO: 20 - RNA, 22 nt, homo sapiens
caucuuacugggcagcauugga

SEQ ID NO: 21 - RNA, 22 nt, homo sapiens
uggaauguaaggaagugugugg

SEQ ID NO: 22 - RNA, 22 nt, homo sapiens
auaagacgagcaaaaagcuugu

SEQ ID NO: 23 - RNA, 22 nt, homo sapiens
ugccugucuacacuugcugugc

SEQ ID NO: 24 - RNA, 22 nt, homo sapiens
aaaucucugcaggcaaauguga

SEQ ID NO: 25 - RNA, 21 nt, homo sapiens
ccacaccguaucugacacuuu

SEQ ID NO: 26 - RNA, 21 nt, homo sapiens
caagucacuagugguuccguu

SEQ ID NO: 27 - RNA, 21 nt, homo sapiens
aucacauugccagggauuucc

SEQ ID NO: 28 - RNA, 21 nt, homo sapiens
aucacauugccagggauuacc

SEQ ID NO: 29 - RNA, 21 nt, homo sapiens
uucaaguaauucaggauaggu

SEQ ID NO: 30 - RNA, 22 nt, homo sapiens
aaggagcucacagucuauugag

SEQ ID NO: 31 - RNA, 22 nt, homo sapiens
uguaaacauccccgacuggaag

SEQ ID NO: 32 - RNA, 22 nt, homo sapiens
uguaaacauccuugacuggaag

SEQ ID NO: 33 - RNA, 22 nt, homo sapiens
cuggcccucucugcccuuccgu

SEQ ID NO: 34 - RNA, 23 nt, homo sapiens
ucaagagcaauaacgaaaaaugu

SEQ ID NO: 35 - RNA, 22 nt, homo sapiens
caauguuuccacagugcaucac

SEQ ID NO: 36 - RNA, 23 nt, homo sapiens
ugucugcccgcaugccugccucu

SEQ ID NO: 37 - RNA, 23 nt, homo sapiens
aggcaguguaguuagcugauugc

SEQ ID NO: 38 - RNA, 22 nt, homo sapiens
acucaaaaugggggcgcuuucc

SEQ ID NO: 39 - RNA, 21 nt, homo sapiens
gguggauauuccuucuauguu

SEQ ID NO: 40 - RNA, 22 nt, homo sapiens
agcgagguugcccuuuguauau

SEQ ID NO: 41 - RNA, 21 nt, homo sapiens
aauauaacacagauggccugu

SEQ ID NO: 42 - RNA, 23 nt, homo sapiens
acuucaccugguccacuagccgu

SEQ ID NO: 43 - RNA, 23 nt, homo sapiens
ugaggggcagagagcgagacuuu

SEQ ID NO: 44 - RNA, 22 nt, homo sapiens
aucaugaugggcuccucggugu

SEQ ID NO: 45 - RNA, 22 nt, homo sapiens
ucaggcucaguccccucccgau

SEQ ID NO: 46 - RNA, 22 nt, homo sapiens
aaucguacagggucauccacuu

SEQ ID NO: 47 - RNA, 22 nt, homo sapiens
caaccuggaggacuccaugcug

SEQ ID NO: 48 - RNA, 23 nt, homo sapiens
aggaccugcgggacaagauucuu

SEQ ID NO: 49 - RNA, 22 nt, homo sapiens
uuguacaugguaggcuuucauu

SEQ ID NO: 50 - RNA, 22 nt, homo sapiens
ugaaacauacacgggaaaccuc

SEQ ID NO: 51 - RNA, 22 nt, homo sapiens
aaugcaccugggcaaggauuca

SEQ ID NO: 52 - RNA, 22 nt, homo sapiens
gggagccaggaaguauugaugu

SEQ ID NO: 53 - RNA, 21 nt, homo sapiens
gugucuuuugcucugcaguca

SEQ ID NO: 54 - RNA, 22 nt, homo sapiens
aucgugcaucccuuuagagugu

SEQ ID NO: 55 - RNA, 22 nt, homo sapiens
gaaagcgcuucccuuugcugga

SEQ ID NO: 56 - RNA, 22 nt, homo sapiens
caaagcgcuccccuuuagaggu

SEQ ID NO: 57 - RNA, 21 nt, homo sapiens
aaagcgcuucccuucagagug

SEQ ID NO: 58 - RNA, 21 nt, homo sapiens
gaaagcgcuucucuuuagagg

SEQ ID NO: 59 - RNA, 22 nt, homo sapiens
aaagugcuucccuuuggacugu

SEQ ID NO: 60 - RNA, 24 nt, homo sapiens
acaaagugcuucccuuuagagugu

SEQ ID NO: 61 - RNA, 22 nt, homo sapiens
aacgcacuucccuuuagagugu

SEQ ID NO: 62 - RNA, 23 nt, homo sapiens
gaacgcgcuucccuauagagggu

SEQ ID NO: 63 - RNA, 22 nt, homo sapiens
aaaaguaauugugguuuuggcc

SEQ ID NO: 64 - RNA, 22 nt, homo sapiens
aaaaguaauugcgguuuuugcc

SEQ ID NO: 65 - RNA, 22 nt, homo sapiens
aaaaguaauugugguuuuugcc

SEQ ID NO: 66 - RNA, 21 nt, homo sapiens
uaauuuuauguauaagcuagu

SEQ ID NO: 67 - RNA, 19 nt, homo sapiens
ugucucugcugggguuucu

SEQ ID NO: 68 - RNA, 22 nt, homo sapiens
ugugucacucgaugaccacugu

SEQ ID NO: 69 - RNA, 21 nt, homo sapiens
ucuaguaagaguggcagucga

SEQ ID NO: 70 - RNA, 21 nt, homo sapiens
aggaggcagcgcucucaggac

SEQ ID NO: 71 - RNA, 23 nt, homo sapiens
ggcagguucucacccucucuagg

SEQ ID NO: 72 - RNA, 22 nt, homo sapiens
cuugguucagggagggucccca

SEQ ID NO: 73 - RNA, 23 nt, homo sapiens
ugucacucggcucggcccacuac

SEQ ID NO: 74 - RNA, 17 nt, homo sapiens
ucucgcuggggccucca

SEQ ID NO: 75 - RNA, 22 nt, homo sapiens
cugcccuggcccgagggaccga

SEQ ID NO: 76 - RNA, 21 nt, homo sapiens
cgcgggugcuuacugacccuu

SEQ ID NO: 77 - RNA, 22 nt, homo sapiens
ucuucucuguuuuggccaugug